(19) United States
(12) Patent Application Publication
(10) Pub. No.: US 2022/0273029 A1
(43) Pub. Date: Sep. 1, 2022

(54) VAPING DEVICE FOR DYNAMIC AEROSOL FORMULATION

(71) Applicant: Bernard Gabriel JUSTER, Netanya (IL)

(72) Inventor: Bernard Gabriel JUSTER, Netanya (IL)

(21) Appl. No.: 17/626,183

(22) PCT Filed: Jul. 12, 2020
(86) PCT No.: PCT/IL20/50779

§ 371 Date: Jan. 11, 2022

Related U.S. Application Data
(60) Provisional application No. 62/872,720, filed on Jul. 11, 2019.

(51) Int. Cl. A24F 40/10; A24F 40/42; A24F 40/50

(52) U.S. Cl. CPC A24F 40/10; A24F 40/42; A24F 40/50

(57) ABSTRACT

A system and device are provided for dynamically adjusting aerosol formulations of vaporized liquid concentrates that may be inhaled by a user. The device may have one or more cartridges, each having a cartridge chamber. The cartridge chamber has a piston at one end that when pressed ejects a liquid concentrate from an opposite end into a heating chamber. An electrical heating signal heats the heating chamber to vaporize the liquid concentrate to generate an aerosol. An actuator of the device drives the piston. The actuator signal and the electrical heating signal are synchronized to eject and to vaporize the same quantity of liquid concentrate. A microcontroller unit may provide the actuator signal and the electrical heating signal according to a formulation of aerosol that may adjusted by a user during operation. The formulation may specify ratios of aerosols from multiple cartridges.

### FIELD OF THE INVENTION

The present invention generally relates to the field of smoking devices.

### BACKGROUND

Certain substances, such as cannabis extracts in the form of liquid concentrates, can be vaporized and inhaled for medical and non-medical purposes. These substances can be combined to produce different formulations. For example, in the case of cannabis, liquid concentrates from different cannabis strains can be mixed to produce different formulations, having different combinations of cannabinoids (including Tetrahydrocannabinol (THC) and Cannabidiol (CBD), as well as terpenes and other extracts). Formulations preferred by a given user may change according to a user's medical requirements or mood. Liquid concentrates for use in “vaping” devices such as electronic cigarettes (e-cigarettes), may be sold with different formulations, typically being provided in disposable or refillable cartridges. For a greater variety of formulations, a user may also buy such “e-liquid” preparations with given formulations and then mix them in a refillable cartridge. However, many users find such self-preparation of formulations to be inconvenient. There is therefore a need for a convenient system and method for a user to adjust formulations of vaporizable liquid concentrates.

To vaporize liquid concentrates, many vaping devices have a wick of absorbent, cotton-like material that brings the concentrate into contact with a heating element. However, a problem may arise of the wick being too dry, that is, the wick may not fully absorb the concentrate, thereby reducing the generation of vapor and potentially damaging the heating element. The wick is also difficult to insert into the device in an automated manufacturing process.

Propylene Glycol (PG) and glycerol (commonly referred to as a “vegetable glycerin” in liquid formulations) are two of the most common vaporizing solvents used in liquid concentrates for e-cigarettes. The present invention is applicable to concentrates including any known extracts and solvents.

### SUMMARY

Embodiments of the present invention provide a system and a vaping device for dynamically altering the formulation of aerosols released by the vaping device (e.g., an e-cigarette), where the aerosols are produced by vaporizing liquid concentrates, such as cannabis extracts. The formulation may include ratios of aerosols that may be adjusted by a user during operation. The vaping device may include multiple cartridges. Each cartridge may include: a cartridge chamber holding a liquid concentrate; a piston at one end of the cartridge chamber, to change the volume of the cartridge chamber and to eject the liquid concentrate from an opposite end of the cartridge chamber; and a heating chamber that receives the liquid concentrate from the cartridge chamber and, upon receiving an electrical heating signal, vaporizes the liquid concentrate within the heating chamber to generate an aerosol. The vaping device may also include multiple actuators, each corresponding to a respective cartridge, each actuator configured to receive an actuator signal and responsively to generate a mechanical force to advance the piston of the respective cartridge.

The vaping device may also include a microcontroller, which has a processor and a memory, the memory including instructions that when executed by the processor cause the microcontroller to perform steps of: receiving, during user operation of the vaping device, a ratio setting, wherein the ratio setting specifies relative amounts of aerosols from each cartridge that are to be combined into a generated aerosol mixture; according to the ratio setting, for each relative aerosol amount required from each cartridge, calculating an amount of liquid concentrate to vaporize from each cartridge, and responsively calculating for each cartridge an actuator signal to cause the corresponding actuator to eject the calculated liquid concentrate amount from the cartridge chamber, and further calculating an electrical heating signal to cause the heating chamber to vaporize the calculated liquid concentrate amount; and transmitting the calculated electrical heating signals and the calculated actuator signals for each cartridge to the respective heating chambers and corresponding actuators of the multiple cartridges to generate an aerosol mixture having the specified ratios of aerosols.

In some embodiments, the liquid concentrate may be ejected from the cartridge chamber to an outlet capillary that leads to the heating chamber. The ratio setting may be received by the device from a mobile device having a dynamic formulation application by which a user enters the ratio setting.

The walls of the heating chambers may be porous, and the aerosol generated by the vaporization of the liquid concentrate in each heating chamber may be released from the heating chamber through the porous walls. The vaping device further includes a mouthpiece, and wherein the aerosols generated from each cartridge are mixed in the mouthpiece before being inhaled by a user.

In some embodiments, the cartridges are replaceable. The device may include a detachable mouthpiece and the cartridges may be accessible for replacement by removing the detachable mouthpiece. The detachable mouthpiece may be affixed to a base of the device by magnetic contacts. The actuators may include piezoelectric motors or stepping motors.

The device of claim 1, wherein the actuator signal and the electrical heating signal have appropriate duty cycles and durations to respectively eject and to vaporize the calculated amount of liquid concentrate. Calculating the duty cycles for delivering the electrical heating and actuating signals for each cartridge may include multiplying the rate of aerosol generation by respective heating and actuating duty cycle factors. The heating and actuating duty cycle factors may be specified for each cartridge, according to a type of the liquid concentrate of the given cartridge. The duty cycle factors may be specified in pulses per volume displaced.

In some embodiments, the ratio setting indicates a rate of aerosol generation for each cartridge by indicating the relative amount of each aerosol as a percent of a total amount of the mixture of aerosols.

Further embodiments of the present invention provide a system for generating an aerosol mixture, wherein ratios of aerosols mixed into the aerosol mixture are user-adjustable, the system comprising the vaping device described above, together with a mobile device having a dynamic formulation application into which a user enters the ratio setting and which transmits the ratio setting to the vaping device.

Further embodiments of the present invention provide a device for generating an aerosol, the device including a disposable cartridge. The disposable cartridge may include: a cartridge chamber holding a liquid concentrate; a piston at one end of the cartridge chamber, configured to advance into the cartridge chamber when a force is applied to the piston and upon advancing to eject the liquid concentrate from an opposite end of the cartridge chamber; and a heating chamber that receives the liquid concentrate from the cartridge chamber and, upon receiving an electrical heating signal, vaporizes the ejected liquid concentrate within the heating chamber to generate an aerosol. The aerosol may be released from the heating chamber through porous walls of the heating chamber. The device may also include an actuator configured to receive an actuator signal and responsively to generate a mechanical force to advance the piston of the cartridge to eject the liquid concentrate. The device may also include a microcontroller having a processor and a memory storing instructions, which when executed by the processor cause the microcontroller to perform steps of: receiving, during user operation of the device, a ratio setting, wherein the ratio setting specifies an amount of an extract in the liquid concentrate to be provided during a given period of time; according to the ratio setting, calculating an amount of liquid concentrate to vaporize from the cartridge, and responsively calculating an actuator signal to cause the actuator to eject the calculated amount of liquid concentrate from the cartridge chamber, and further calculating an electrical heating signal to cause the heating chamber to vaporize the calculated amount of liquid concentrate; and transmitting the calculated electrical heating signal and actuated signal to the respective heating chamber and actuator to generate an aerosol having the specified amount of the extract in the liquid concentrate during the given period of time.

### BRIEF DESCRIPTION OF DRAWINGS

For a better understanding of various embodiments of the invention and to show how the same may be carried into effect, reference will now be made, by way of example, to the accompanying drawings. Structural details of the invention are shown to provide a fundamental understanding of the invention, the description, taken with the drawings, making apparent to those skilled in the art how the several forms of the invention may be embodied in practice. In the figures:

FIG. 1 is a schematic diagram of a liquid concentrate cartridge, for a vaping device for dynamically adjusting aerosol formulations, in accordance with an embodiment of the present invention;

FIG. 2 is a schematic diagram of components, including a liquid concentrate cartridge, of a vaping device for dynamically adjusting aerosol formulations, in accordance with an embodiment of the present invention;

FIG. 3 is a schematic diagram of components, including a mouthpiece and base, of a vaping device for dynamically adjusting aerosol formulations, in accordance with an embodiment of the present invention.

FIG. 4 is a schematic diagram of a vaping system, including a vaping device for dynamically adjusting aerosol formulations, in accordance with an embodiment of the present invention; and

FIG. 5 is a schematic diagram of components, including a heating chamber, of a vaping device for dynamically adjusting aerosol formulations, in accordance with an embodiment of the present invention.

### DETAILED DESCRIPTION

Embodiments of the present invention provide a system and device for dynamically adjusting aerosol formulations of vaporized liquid concentrates that may be inhaled by a user. The device, also referred to herein as a “vaping device,” may include one or more liquid concentrate cartridges that may be refillable, or which may be replaced by a user (e.g., a consumer or patient). A cartridge of the device contains a liquid concentrate (i.e., a “consumable substance”) in a cartridge chamber, which, as with a syringe, has a piston or plunger configured to press on the liquid concentrate from one end of the chamber, forcing concentrate out of a hole at the opposite end. The liquid concentrate ejected from the cartridge chamber enters a heating chamber, where the liquid concentrate is vaporized.

The piston displacement and the temperature of the heater may be synchronized to eject and to vaporize the same amount of liquid concentrate during a given period of time, typically the period time being a period of aerosol inhalation by a user. A microcontroller unit (MCU) may provide an actuator signal to drive the piston displacement and an electrical heating signal to heat the heating chamber according to a formulation of aerosol that may adjusted by a user during operation. The formulation may specify ratios of aerosols from multiple cartridges. The aerosols generated from each cartridge are released into a common mouthpiece, thereby creating an aerosol mixture that includes the multiple aerosols in the desired ratio.

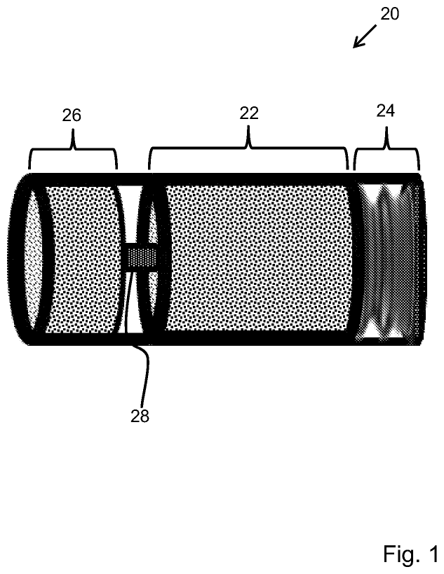

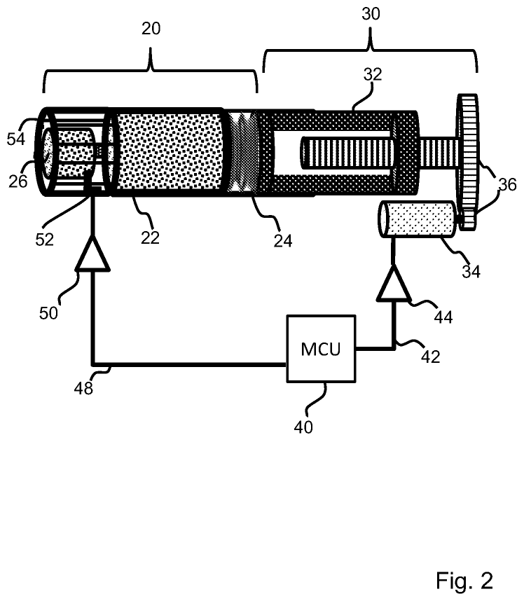

FIG. 1 is a schematic diagram of a liquid concentrate cartridge 20 for a vaping device for dynamically adjusting aerosol formulations, in accordance with an embodiment of the present invention. The cartridge typically has a tubular form (similar to a syringe), having a cartridge chamber 22, which contains the liquid concentrate, and a sliding rear base, or piston 24. The piston can be pushed (i.e., driven inward) by a linear actuator, described below with respect to FIG. 2. When the piston is pushed inward, the volume of the cartridge chamber is reduced, forcing the liquid concentrate to be ejected out from the other end of the cartridge chamber 22 into a heating chamber 26. As described herein, the heating chamber 26 vaporizes the liquid concentrate that is inside the heating chamber, creating an aerosol that is inhaled by a user. Typically, the heating chamber is a ceramic heater with a cavity into which the liquid concentrate flows. As described further herein, the heating chamber typically has porous walls, and the aerosol generated by vaporization typically escapes from the heating chamber through the porous walls (rather than through a single outlet hole).

Typically, the cartridge chamber and the heating chamber are connected to each other by an outlet capillary 28, typically a thin metal tube. Prior to a user's initial “puff,” i.e., first inhalation during a smoking session, the heating chamber can be “primed” by filling its internal cavity with the liquid concentrate. An initial electrical current (i.e., an “electrical signal”) applied to the heating chamber at this initial stage may also heat the outlet capillary 28, lowering the viscosity of the liquid concentrate flowing through it, to permit easier flow from the cartridge chamber to the heating chamber.

As described further herein, the cartridge 20 is typically replaceable, meaning that it may be inserted into a vaping device when the cartridge chamber is full of liquid concentrate, and may then be replaced when the liquid concentrate has been consumed (i.e., the cartridge may be considered “disposable”). When the cartridge 20 is placed inside a vaping device, the cartridge fits into place such that the piston is in contact with a linear actuator that operates the piston, as described with respect to FIG. 2.

FIG. 2 is a schematic diagram of the liquid concentrate cartridge 20 in contact with an actuator 30 of a vaping device for dynamically adjusting aerosol formulations, in accordance with an embodiment of the present invention. The actuator 30, which is typically a “linear actuator,” includes a plunger 32 that drives the cartridge piston 24, in order to eject liquid concentrate from the cartridge chamber 22 into the heating chamber 26.

The plunger 32 moves linearly, both forward and backwards. The plunger moves forward (hereinbelow, the “positive” direction) when pressing the cartridge piston into the cartridge chamber to eject the liquid concentrate. The plunger moves backwards (the “negative” direction) to return to its starting position when a user replaces a cartridge, inserting a new cartridge that typically has a full cartridge chamber (meaning the piston is in an initial position where it has not yet been pressed inwards). The plunger is typically driven by a motor 34, through a transmission mechanism, such as a linear gear train 36, or other similar linear drive mechanism known in the art. For example, the plunger may be advanced by a screw shaft of the gear train 36, as indicated in the figure. The motor 34 may incorporate different motor technologies known in the art; for example, the motor may be a stepping motor or piezoelectric motor.

As described above, when the cartridge 20 is placed inside a vaping device, the cartridge piston 24 is fit against the actuator plunger 32.

Movement of the actuator 30 is controlled by a microcontroller unit (MCU) 40, which issues an actuator signal 42 to the actuator motor 34, to advance the plunger by increments, according to the amount of liquid concentrate that is to be ejected. An actuator signal driver 44, between the MCU 40 and the actuator motor 34 may be employed to boost or otherwise modify the actuator signal as appropriate to drive the actuator motor. The MCU 40 may, for example issue two types of actuator signals, one to trigger positive (i.e., “inward” or “forward”) linear movement and the second to trigger negative (“backward”) linear movement. Responsively, the driver 44 may be configured to convert the two types of actuator signals into actuator signals having positive or negative polarity to drive the actuator motor 34 in the respective forward or backward directions. The “negative” signal may be issued by the MCU when, for example, a cartridge is being replaced, so as to cause the actuator plunger to return to its initial position.

The actuator 30 may also include a force sensor that may provide a signal to the MCU 40 indicating a force of applying the plunger 32 to the cartridge piston 24. For example, if a new cartridge has previously been partially emptied, the force sensor may indicate to the MCU 40 that the plunger 32 at its initial position is not in contact with the cartridge piston 24. The MCU may then advance the actuator until the plunger comes into contact with the cartridge piston 24. The force sensor may be a type of pressure or stress sensor known in the art, which may be positioned, for example, on the face of plunger, where the plunger contacts the piston. Alternatively, the force sensor may be a sensor of the current applied to the motor, which may be an indicator of the force required to drive the gear train.

The force applied to the plunger 32 by the motor 34 may be a function of parameters such as viscosity of the liquid concentrate, friction of an inner wall of the cartridge, etc.

The MCU 40 also provides an electrical heating signal 48 to the heating chamber 26, to cause the heating chamber to heat up and vaporize the liquid concentrate that is contained within it. The electrical heating signal may also be driven by an electrical heating signal driver 50, for example to increase the voltage provided by the MCU 40, because the voltage required to heat the heating chamber to a vaporization is typically higher than the voltage provided by the MCU.

The heating chamber also includes an electrical contact 52 to receive the electrical heating signal when the cartridge is installed in a vaping device as described below.

In one embodiment, the heating chamber may be surrounded by a cage-like safety rail 54 in order to prevent users from touch a hot heating chamber when replacing the cartridge. Typically, the heating chamber is not exposed during operation, but is covered by a mouthpiece of the vaping device, as described below. The safety rail 54 is structured so that vapor can easily pass through it.

Typically, cartridges are provided with identifying information that includes a product identifier with respect to the type of liquid concentrate in the container, and may include additional parameters, such as appropriate vaporization temperatures. In one embodiment of the present invention, a replaceable cartridge as described above, may be installed in a vaping device, including an actuator as described above, such that the cartridge piston is put in contact with the actuator plunger.

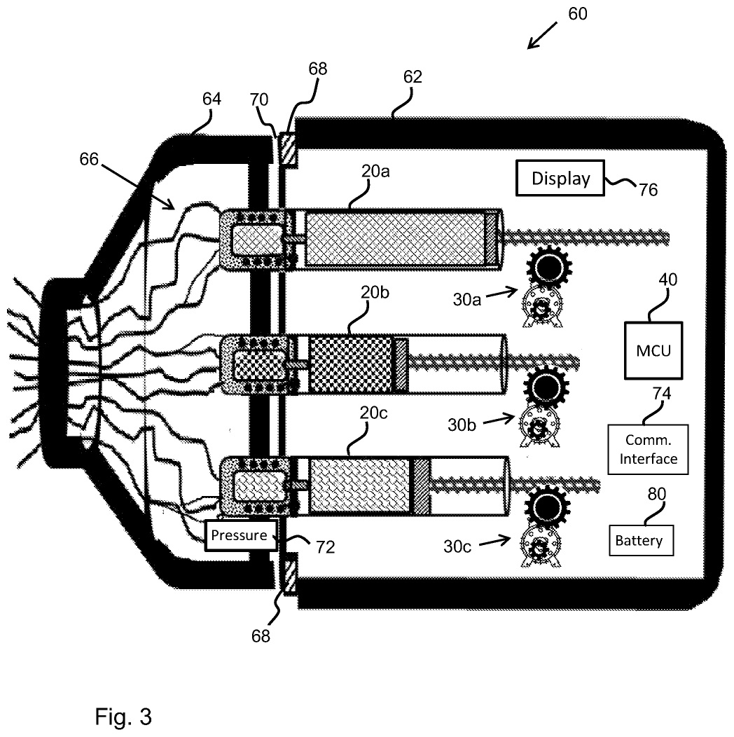

FIG. 3 is a schematic diagram of a vaping device 60 for dynamically adjusting aerosol formulations, in accordance with an embodiment of the present invention. The vaping device 60 is a multi-cartridge device, such that aerosols of the multiple cartridges are mixed, and the ratios of the aerosols in the mixture are user-adjustable, as described further hereinbelow. Three cartridges, 20a, 20b, and 20c, are indicated as being in the exemplary vaping device shown in the figure. Vaping devices provided by the present invention may have one or multiple cartridges, each installed in a respective cylindrical slot of the device body, or “base” 62. Having three cartridges permits the mixing of aerosols from three different types liquid concentrates, which may be, for example, a high CBD formulation, a high THC formulation, and a Terpene-rich formulation, accordingly.

The cartridges are shown as inserted into respective slots of the device base 62, the cartridges when inserted being positioned in contact with respective actuators 30a, 30b, and 30c. The base 62 is affixed to a detachable mouthpiece 64. The mouthpiece is typically detached to allow replacement of the cartridges and then affixed to the base. The cartridges may be locked in place by locking mechanisms known in the art, such as a spring locking mechanism, and/or may be held in place by the mouthpiece. In some embodiments, an outer shell of the mouthpiece is transparent, for example, glass, such that a user may view the generation of aerosols.

As indicated in the figure, the heating chambers of the cartridges extend beyond the base into the mouthpiece. Aerosols generated from each of the heating chambers are released into and mixed in the mouthpiece 64, so that the user may inhale an aerosol mixture 66 that is a combination of the separately generated aerosols.

The mouthpiece 64 may be affixed to the base by connectors 68, which may, for example, be magnetic connectors. The mouthpiece may be configured with air vents 70 to provide air intake.

Inhalation by a user may be detected by a pressure sensor 72., which may transmit an indicative pressure signal to the MCU 40. The MCU may cause the liquid concentrate to be injected into the heating chamber, and to be vaporized in the heating chamber, upon sensing inhalation by a user. The aerosols (i.e., vapors) generated by vaporization are mixed in the mouthpiece and then inhaled by the user.

Some of the additional elements of a typical vaping device 60 of the present invention are also indicated in the figure. These include, for example, a communications interface 74, which enables data transfer to and from external devices, such as a mobile device as described below. The vaping device may also include a display 76, which may be, for example, a textual or graphic display, or one or more lights (e.g., LEDs), which may provide visual indicators of various operating states, such as an indicator that the vaping device is connecting wirelessly to a mobile device (or “scanning” for a mobile device).

The vaping device typically also includes a rechargeable and/or replaceable battery 80, which provides power to the other components, such as the MCU 40, the communications interface 74, the display 76, the actuators 30, and the electrical drivers 44 and 50.

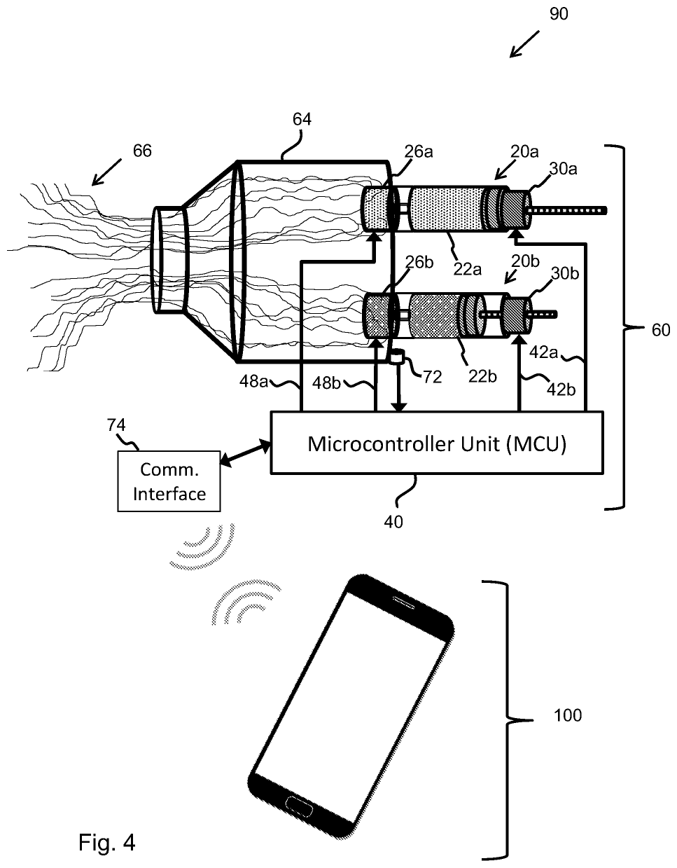

FIG. 4 is a schematic diagram of a vaping system 90, including a vaping device 60 for dynamically adjusting aerosol formulations, in accordance with an embodiment of the present invention. Shown are two cartridges in the exemplary vaping device of the figure, cartridges 20a and 20b. Cartridge 20a has a corresponding actuator 30a; cartridge 20b has a corresponding actuator 30b. Also indicated are the respective heating chambers, 26a and 26b, of the cartridges 20a and 20b. As shown, the heating chambers release aerosols to generate an aerosol mixture 66 in the mouthpiece 64, as described above.

System 90 also includes a mobile device 100, which communicates with the MCU 40 of the vaping device 60 by means of the communications interface 74. Typically, the communications will be configured as wireless communications, such as by Bluetooth or Wi-Fi Direct.

The mobile device 100 typically includes a user application (an “aerosol formulation application”) that allows the user to specify an aerosol formulation, by means of which a user specifies ratios of aerosols of the different cartridges that are to be mixed to create the aerosol mixture 66. The user typically specifies (or scans) identifying information regarding the cartridges that are currently installed in the vaping device. This identifying information may then be displayed in the aerosol formulation application, and the user may then specify ratios of the different cartridge aerosols (i.e., define a “ratio setting”), for example, by specifying a percent of each type of aerosol to be included in the combined mixture of aerosols, or a weight amount of any given extract. Alternatively, the user may set ratios of main ingredients (such as CBD and THC), which may be present in different amounts in the different installed cartridges. Alternatively, the user may set a total amount (by weight or volume) of one or more types of ingredients, such as CBD extract, to be consumed during a session. This option of limiting the total amount of one or more ingredients or extracts may also be applied in a vaping device having only a single cartridge, as described further below.

Hereinbelow, any of these types of settings regarding the ratios or amounts of aerosols or ingredients are referred to by the term, “ratio setting.”

The user may change the ratio before or during a smoking session, thereby facilitating dynamic alteration of the aerosol formulation. In addition to the ratio setting, the vaping system may also be configured to permit the user to dynamically configure additional settings, such as a maximum amount of aerosol to be produced by any given type of cartridge, as well a period of time over which the amount should be released.

Upon receiving the ratio setting from the mobile device, as well as any additional settings described above, the MCU 40 may calculate an amount of liquid concentrate to vaporize from each cartridge, for each user inhalation (i.e., “puff”). The calculation of liquid concentrate amounts to be vaporized may depend on parameters of each specific liquid concentrate. The calculation may be based on results of testing of the different liquid concentrates that may be used in the vaping device.

After calculating the amount of liquid concentrate to vaporize for each cartridge, the MCU then determines an actuator signal to transmit to each actuator to eject the calculated liquid concentrate amount from each corresponding cartridge chamber. That is, the MCU calculates and transmits an actuator signal 42a to the actuator 30a, and an actuator signal 42b to the actuator 30b. The MCU also calculates respective electrical heating signals 48a and 48b to cause the heating chambers 26a and 26b respectively to vaporize the calculated liquid concentrate amount that is ejected from the respective cartridge chambers 22a and 22b. That is, the MCU synchronizes the liquid concentrate ejection with the liquid concentrate vaporization. The amount of liquid concentrate vaporized in a heating chamber is a function of the temperature of the chamber during vaporization and the period of time that the vaporizing temperature is maintained.

To modify the amount of liquid concentrate vaporized during a given period of time, the waveforms of the actuator signals and the electrical heating signals for each cartridge may be modified. For example, to achieve a 2:1 ratio between two cartridges (e.g., cartridges 20a and 20b in FIG. 4), the cartridge set to the higher rate (i.e., the cartridge set to emit twice the amount of the other cartridge) may be operated with signals at a “full continuous regimen,” meaning constant signals (e.g., pulse-width modulation (PWM) or DC voltages). The cartridge set to the lower rate may be operated by turning off the signals for 250 milliseconds for every 500 millisecond period. The reduced effective power of the signals would thus achieve an effective aerosol mass reduction of 50% over a puff duration. That is, the actuator of the cartridge set to the lower rate would eject half as much of the liquid concentrate from the cartridge chamber, relative to the amount ejected by the higher rate cartridge, and, correspondingly, the heating chamber would vaporize half as much liquid concentrate relative to the amount vaporized by the higher rate cartridge. Different ratio amounts may be achieved by having different ratio duty cycles (i.e., ratios of “on” and “off” pulses), for the actuator signals and the electrical heating signals.

More generally, different types of liquid concentrate may also require different “regimens” of actuating and heating signals to produce given amounts of aerosols. Heating and actuating duty cycle factors may be specified for each type of cartridge, according to a type of the liquid concentrate of the given cartridge. The duty cycles for delivering the electrical heating and actuating signals may be calculated, for example, by multiplying the rate of aerosol generation needed to achieve a given ratio setting by respective heating and actuating duty cycle factors of a given cartridge. The duty cycle factors may be specified in pulses per volume displaced or vaporized. Duty cycles of the electrical heating and actuating signals for a given cartridge may be different, but are synchronized to achieve ejection and vaporization of the same amount of liquid concentrate.

The MCU may also use resistance sensing of a heating element of the heating chamber, to ensure that the temperatures are not too high or too low (which would result in inaccurate amounts of vaporization). As described above, it is to be understood that if a vaping device includes only a single cartridge, the MCU may be configured to receive a ratio setting in the form of a maximum quantity of an extract to supply during a session, and responsively to determine synchronized electrical heating and actuating signals for the cartridge to achieve appropriate liquid concentrate vaporization with each inhalation.

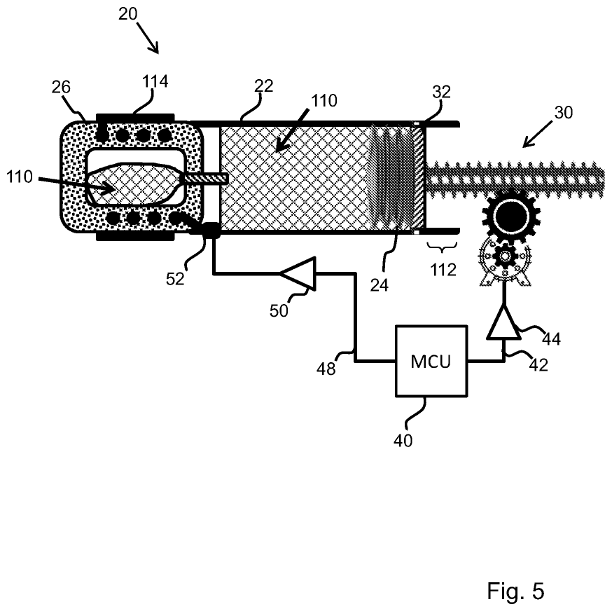

FIG. 5 is a schematic diagram of components, including a heating chamber, of a vaping device for dynamically adjusting aerosol formulations, in accordance with an embodiment of the present invention. Shown in the figure are the components of the actuator 30 corresponding to the cylinder 20, including the actuator plunger 32 in contact with the cylinder piston 24. In addition, a cross section of the cylinder chamber 22 is shown, including liquid concentrate 110, shown flowing from the cylinder chamber into the heating chamber 26. As shown, the amount of the liquid concentrate 110 that flows into the heating chamber is determined by the linear displacement 112 of the actuator plunger 32, which in turn is determined by the actuator signal 42 from the MCU 40, as driven by the actuator signal driver 44.

In the cross-section of the heating chamber shown, the porous walls of the heating chamber are indicating as including a heating coil 114, which may be a metal coil or any other type of resistive coil. The heating coil may be electrically grounded to the device base, with the positive lead connected the electrical contact 52, by which the electrical heating signal 48 is received. Upon installing the cartridge into a vaping device, the heating coil is connected to device ground and to the output of its respective electrical heating signal driver 50.

Unlike vaping devices that determine a dose of aerosol to deliver based on fixed assumptions regarding inhalation rates and inhalation intensity, the present invention provides a more precise calculation of dosage delivered by measuring the inhalation pressure to determine when the user is actually inhaling, combined with a self-calibration algorithm that determines the amount of aerosol being generated by vaporization over time.

The following table includes definitions of the numeric indicators used in the figures:

| 20, 20a, 20b, 20c | cartridge |
|---|---|
| 22, 22a, 22b | cartridge chamber (with liquid concentrate) |
| 24 | cartridge piston (″piston″) |
| 26, 26a, 26b | heating chamber |
| 28 | outlet capillary of cartridge chamber |
| 30, 30a, 30b, 30c | linear actuator (″actuator″) |
| 32 | actuator plunger |
| 34 | actuator motor |
| 36 | actuator gear train (″transmission″) |
| 40 | microcontroller unit (MCU) |
| 42, 42a, 42b | actuator signal (received by actuator from MCU) |
| 44 | actuator signal driver |
| 48, 48a, 48b | electrical heating signal (received by heating chamber from MCU) |
| 50 | electrical heating signal driver |
| 52 | heating chamber electrical contact |
| 54 | heating chamber cage-like safety rail |
| 60 | multi-cartridge, vaping device (″device″) |
| 62 | device base |
| 64 | device mouthpiece |
| 66 | generated aerosol |
| 68 | mouthpiece connectors |
| 70 | air vent |
| 72 | pressure sensor (user inhalation sensor) |
| 74 | communications interface (typically wireless) |
| 76 | device display (and or LEDs) |
| 80 | battery |
| 90 | vaping system for dynamic aerosol formulation (including vaping device and mobile device) |
| 100 | mobile device (including dynamic formulation application) |
| 110 | liquid concentrate |
| 112 | linear displacement of actuator (e.g., during |
| | one puff) |
| 114 | heating coils of heating chamber |

Processing elements of the system described herein may be implemented in digital electronic circuitry, or in computer hardware, firmware, software, or in combinations thereof. Such elements can be implemented as a computer program product, tangibly embodied in an information carrier, such as a non-transient, machine-readable storage device, for execution by, or to control the operation of, data processing apparatus, such as a programmable processor, computer, or deployed to be executed on multiple computers at one site or one or more across multiple sites. Memory storage for software and data may include multiple one or more memory units, including one or more types of storage media. Examples of storage media include, but are not limited to, magnetic media, optical media, and integrated circuits such as read-only memory devices (ROM) and random access memory (RAM). Network interface modules may control the sending and receiving of data packets over networks.

Mobile devices may be any computing device permitting user input to interactive applications as described above.

Method steps associated with the system and process can be rearranged and/or one or more such steps can be omitted to achieve the same, or similar, results to those described herein.

It is to be understood that the embodiments described hereinabove are cited by way of example, and that the present invention is not limited to what has been particularly shown and described hereinabove.

1. A device generating an aerosol mixture, wherein ratios of aerosols mixed into the aerosol mixture are user-adjustable, the device comprising:
multiple cartridges, wherein each cartridge comprises: a cartridge chamber holding a liquid; a piston at one end of the cartridge chamber, to change the volume of the cartridge chamber and to eject the liquid from an opposite end of the cartridge chamber; and a heating chamber that receives the liquid from the cartridge chamber and, upon receiving an electrical heating signal, vaporizes the liquid within the heating chamber to generate an aerosol;
multiple actuators, each corresponding to a respective cartridge, each actuator configured to receive an actuator signal and responsively to generate a mechanical force to advance the piston of the respective cartridge; and
a microcontroller, comprising a processor and memory storing instructions that when executed by the processor cause the microcontroller to perform steps of:
receiving, during user operation of the device, a ratio setting, wherein the ratio setting specifies relative amounts of aerosols from each cartridge that are to be combined into a generated aerosol mixture;
according to the ratio setting, for each relative aerosol amount required from each cartridge, calculating an amount of liquid to vaporize from each cartridge, and responsively calculating for each cartridge an actuator signal to cause the corresponding actuator to eject the calculated amount of liquid from the cartridge chamber, and further calculating an electrical heating signal to cause the heating chamber to vaporize the calculated amount of liquid; and
transmitting the calculated electrical heating signals and the calculated actuator signals for each cartridge to the respective heating chambers and corresponding actuators of the multiple cartridges to generate an aerosol mixture having the specified ratios of aerosols.

2. The device of claim 1, wherein the liquid is ejected from the cartridge chamber to an outlet capillary that leads to the heating chamber.

3. The device of claim 1, wherein the ratio setting is received by the device from a mobile device having a dynamic formulation application by which a user enters the ratio setting.

4. The device of claim 1, wherein the walls of the heating chambers are porous, and the aerosol generated by the vaporization of the liquid in each heating chamber is released from the heating chamber through the porous walls.

5. The device of claim 1, wherein the device further comprises a mouthpiece, and wherein the aerosols generated from each cartridge are mixed in the mouthpiece before being inhaled by a user.

6. The device of claim 1, wherein the cartridges are replaceable.

7. The device of claim 6 wherein the device further comprises a detachable mouthpiece affixed to a base of the device by magnetic contacts, and wherein the cartridges are accessible for replacement by removing the detachable mouthpiece.

8. (canceled)

9. The device of claim 1, wherein the actuators comprise piezoelectric motors and/or stepping motors.

10. (canceled)

11. The device of claim 1, wherein the actuator signal and the electrical heating signal have appropriate duty cycles and durations to respectively eject and to vaporize the calculated amount of liquid.

12. The device of claim 11, wherein calculating the duty cycles for delivering the electrical heating and actuating signals for each cartridge comprises multiplying the rate of aerosol generation by respective heating and actuating duty cycle factors, wherein the heating and actuating duty cycle factors are specified for each cartridge, according to a type of the liquid of the given cartridge.

13. The device of claim 11, wherein the duty cycle factors are specified in pulses per volume displaced.

14. The device of claim 1, wherein the ratio setting indicates a rate of aerosol generation for each cartridge by indicating the amount of each aerosol as a percent of a total volume of the mixture of aerosols.

15. A system for generating an aerosol mixture, wherein ratios of aerosols mixed into the aerosol mixture are user-adjustable, the system comprising:
a vaping device comprising:
multiple cartridges, wherein each cartridge comprises: a cartridge chamber holding a liquid; a piston at one end of the cartridge chamber, to change the volume of the cartridge chamber and to eject the liquid from an opposite end of the cartridge chamber; and a heating chamber that receives the liquid from the cartridge chamber and, upon receiving an electrical heating signal, vaporizes the liquid within the heating chamber to generate an aerosol;
multiple actuators, each corresponding to a respective cartridge, each actuator configured to receive an actuator signal and responsively to generate a mechanical force to advance the piston of the respective cartridge; and
a microcontroller, comprising a processor and memory storing instructions that when executed by the processor cause the microcontroller to perform steps of:
receiving, during user operation of the vaping device, a ratio setting, wherein the ratio setting specifies ratios of aerosols to mix from the liquid of the multiple cartridges;
according to the ratio setting, for each relative aerosol amount required from each cartridge, calculating an amount of liquid to vaporize from each cartridge, and responsively calculating for each cartridge an actuator signal to cause the corresponding actuator to eject the calculated amount of liquid from the cartridge chamber, and further calculating an electrical heating signal to cause the heating chamber to vaporize the calculated amount of liquid; and
transmitting the calculated actuator signals and electrical heating signals for each cartridge to the respective heating chambers and corresponding actuators of the multiple cartridges to generate an aerosol mixture having the specified ratios of aerosols; and
wherein the vaping system further comprises a mobile device having a dynamic formulation application into which a user enters the ratio setting and which transmits the ratio setting to the vaping device.

16. A device for generating an aerosol, the device comprising:
a disposable cartridge comprising: a cartridge chamber holding a liquid; a piston at one end of the cartridge chamber, configured to advance into the cartridge chamber when a force is applied to the piston and upon advancing to eject the liquid from an opposite end of the cartridge chamber; and a heating chamber that receives the liquid from the cartridge chamber and, upon receiving an electrical heating signal, vaporizes the ejected liquid within the heating chamber to generate an aerosol, and wherein the aerosol is released from the heating chamber through porous walls of the heating chamber; and
an actuator configured to receive an actuator signal and responsively to generate a mechanical force to advance the piston of the cartridge to eject the liquid.

17. The device of claim 16, further comprises a microcontroller having a processor and a memory storing instructions, which when executed by the processor cause the microcontroller to perform steps of:
receiving, during user operation of the device, a ratio setting, wherein the ratio setting specifies an amount of an extract in the liquid to be provided during a given period of time;
according to the ratio setting, calculating an amount of liquid to vaporize from the cartridge, and responsively calculating an actuator signal to cause the actuator to eject the calculated amount of liquid from the cartridge chamber, and further calculating an electrical heating signal to cause the heating chamber to vaporize the calculated amount of liquid; and
transmitting the calculated electrical heating signal and actuated signal to the respective heating chamber and actuator to generate an aerosol having the specified amount of the extract in the liquid during the given period of time.
